(19) United States
(12) Patent Application Publication
(10) Pub. No.: US 2023/0138188 A1
(43) Pub. Date: May 4, 2023

(54) SECURE COMPUTER-BASED PRE-OPERATIVE ASSESSMENT

(71) Applicant: United States Government as Represented by the Department of Veterans Affairs, Washington, DC (US)

(72) Inventors: Alon Y. Ben-Ari, Carmichael, CA (US); Sigal Ben-Ari, Carmichael, CA (US)

(21) Appl. No.: 17/980,414

(22) Filed: Nov. 3, 2022

Related U.S. Application Data
(60) Provisional application No. 63/275,275, filed on Nov. 3, 2021.

(51) Int. Cl. G16H 10/60; G16H 20/00; G06F 21/31

(52) U.S. Cl. CPC G16H 10/60; G16H 20/00; G06F 21/31

(57) ABSTRACT

Computing systems, computing devices, computer-implemented methods, and computer-program products are provided for a secure computer-based pre-operative assessment. In some embodiments, a computing system can authenticate a user identifier via an authentication service. Based on authenticating the user identifier, the computing system can cause output of a graphical user interface configured to elicit one or more responses associated with a user condition. The computing system also can receive the one or more responses via the graphical user interface, and can associate the one or more responses with the user identifier. The computing system can generate, based on the one or more responses, at least one of a visual representation or an aural representation indicative of the user condition, and can cause output of at least one of the visual representation or the aural representation.

### CROSS-REFERENCE TO RELATED APPLICATION

This application claims the benefit of and priority to U.S. Provisional Patent Application No. 63/275,275, filed Nov. 3, 2021, the content of which application is hereby incorporated by reference herein in its entirety.

### BACKGROUND

The pre-operative assessment is a process that can identify comorbidities that may lead to patient complications during the anesthetic, surgical, and/or post-operative period. Patients scheduled for elective procedures can attend a pre-operative assessment before the date of their surgery. It is time consuming for patient's clinical staff and requires an additional visit to the hospital.

Although it is advisable for some patients to meet with an anesthesiologist pre-operative, it is not a necessary step for a significant portion of patients undergoing procedures. Screening patients that may require more hands-on approach can enhance delivery of quality and timely care.

### SUMMARY

It is to be understood that both the following general description and the following detailed description are illustrative and explanatory only and are not restrictive.

Embodiments of this disclosure include computing system, computing devices, computer-implemented methods, and computer-program products that, individually or in combination, provide a secure computer-based pre-operative assessment. More specifically, yet not exclusively, embodiments of this disclosure include a secure software application that can allow authentication of a patient using an authentication service. After being authenticated the patient can be presented with a pre-operative survey that can include YES/NO questions and/or other queries. The assessment data can be retained in a secure storage and can be managed by a server device (such as a web server) in compliance with Health Insurance Portability and Accountability Act (HIPAA). Components of the secure software application can supply visual and/or aural representations of assessment data to client devices used by clinical staff.

Although embodiments of this disclosure are described in connection with pre-operative phase of surgical procedures, the disclosure is not limited in that respects. Indeed, the principles and practical applications of this disclosure can be directed to any preliminary phases of an event that can benefit from screening of participants. That event can be a sports event, an academic event (such as application of a standardized test), a chartered travel event, or similar.

Additional elements or advantages of this disclosure will be set forth in part in the description which follows, and in part will be apparent from the description, or may be learned by practice of the subject disclosure. The advantages of the subject disclosure can be attained by means of the elements and combinations particularly pointed out in the appended claims.

This summary is not intended to identify critical or essential features of the disclosure, but merely to summarize certain features and variations thereof. Other details and features will be described in the sections that follow. Further, both the foregoing general description and the following detailed description are illustrative and explanatory only and are not restrictive of the embodiments of this disclosure.

### BRIEF DESCRIPTION OF THE DRAWINGS

The annexed drawings are an integral part of the disclosure and are incorporated into the subject specification. The drawings illustrate example embodiments of the disclosure and, in conjunction with the description and claims, serve to explain at least in part various principles, elements, or aspects of the disclosure. Embodiments of the disclosure are described more fully below with reference to the annexed drawings. However, various elements of the disclosure can be implemented in many different forms and should not be construed as limited to the implementations set forth herein. Like numbers refer to like elements throughout.

FIG. 1A illustrates a non-limiting example of a computing system for a secure computer-based pre-operative assessment, in accordance with one or more embodiments of the disclosure.

FIG. 1B illustrates a non-limiting example of data flow for secure computer-based pre-operative assessment, in accordance with one or more embodiments of the disclosure.

FIG. 1C illustrates another non-limiting example of a computing system for a secure computer-based pre-operative assessment, in accordance with one or more embodiments of the disclosure.

FIG. 2A illustrates a non-limiting example of a user interface, in accordance with one or more embodiments of the disclosure.

FIG. 2B illustrates a non-limiting example of another user interface, in accordance with one or more embodiments of the disclosure.

FIG. 2C illustrates a non-limiting example of yet another user interface, in accordance with one or more embodiments of the disclosure.

FIG. 3A illustrates a non-limiting example of graphical user interface (GUI), in accordance with one or more embodiments of the disclosure.

FIG. 3B illustrates a non-limiting example of a sequence of user interface having textual elements, in accordance with one or more embodiments of the disclosure.

FIG. 4 illustrates a non-limiting example of a graphical representation of responses pertaining to a secure computer-based pre-operative assessment, in accordance with one or more embodiments of the disclosure.

FIG. 5 illustrates a non-limiting example of another graphical representation of responses pertaining to a secure computer-based pre-operative assessment, in accordance with one or more embodiments of the disclosure.

FIG. 6 illustrates a non-limiting example of a computing system for a secure computer-based pre-operative assessment in accordance with one or more embodiments of the disclosure.

FIG. 7 illustrates a non-limiting example of a method for a secure computer-based pre-operative assessment, in accordance with one or more embodiments of the disclosure.

### DETAILED DESCRIPTION

The disclosure recognizes and addresses the issue of evaluation of a subject scheduled for a surgical procedure or another type of event. As mentioned, embodiments of this disclosure include computing devices, computer-implemented methods, and computer-program products that, individually or in combination, can provide a secure computer-based pre-operative assessment. The embodiments of this disclosure are not limited to pre-operative assessments, and can be applied to other types of assessments, such as neuropsychological assessments.

Embodiments of this disclosure provide several advantages over existing technologies and protocols for pre-operative assessment. In a non-limiting example, embodiments of the disclosure can save time for patients and clinical staff alike, while allowing to flag issues with patients in a timely fashion. The time saving is several fold: (1) Obviates the need for a face-to-face appointment, freeing clinical staff to do other tasks; and (2) allows for increased operating room efficiency by reducing the risk for same day case cancellation.

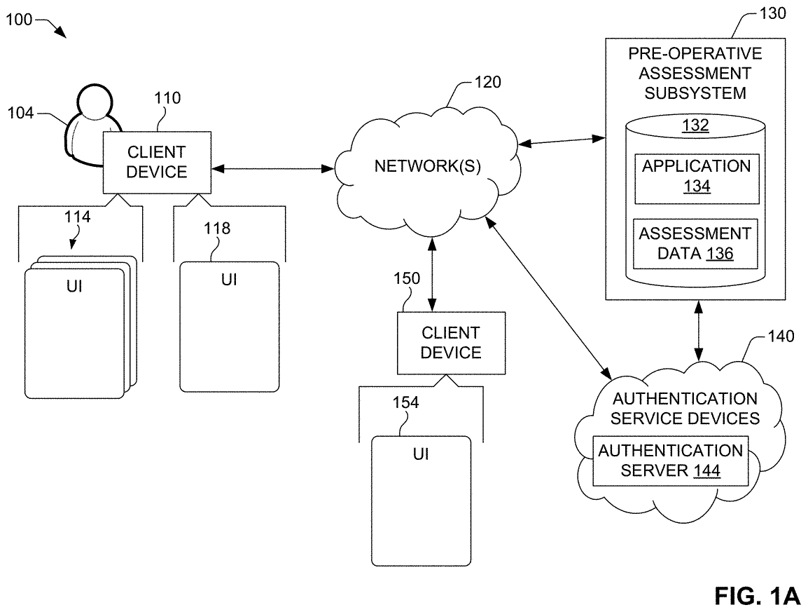

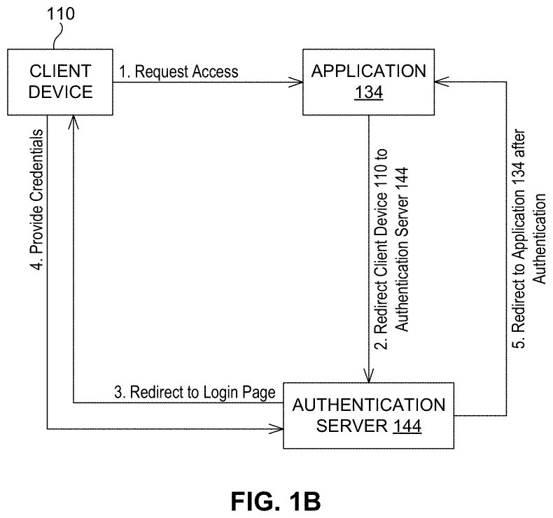

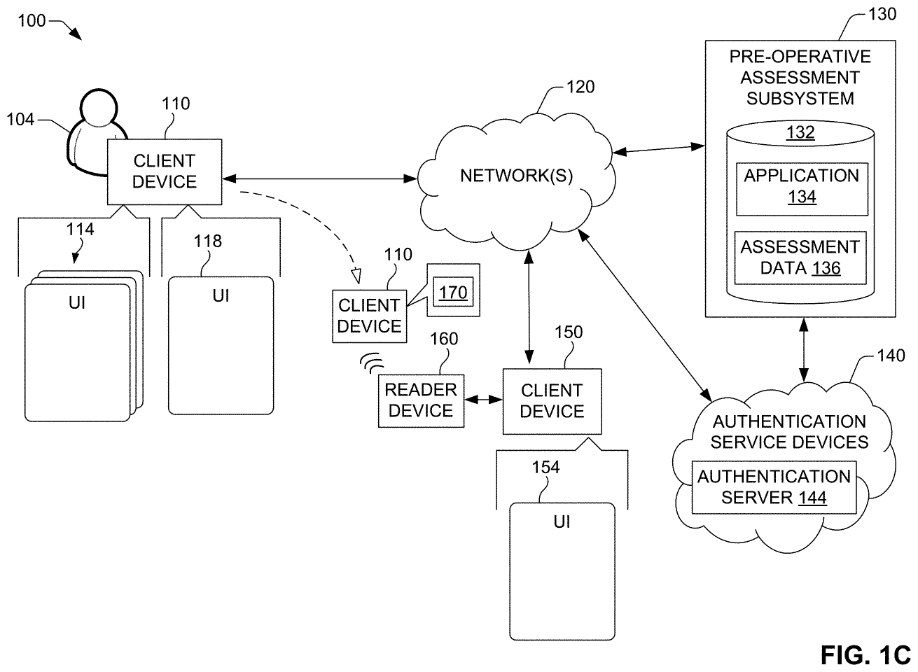

With reference to the drawings, FIG. 1A illustrates a non-limiting example of an computing system 100 for a secure computer-based pre-operative assessment, in accordance with one or more embodiments of the disclosure. The computing system 100 includes a client device 110 that can be operated by a subject 104. The subject 104 can be a Veteran or another individual scheduled to undergo surgery at a future time, for example. The client device 110 can be embodied in, for example, a personal computer, a laptop computer, an electronic-reader (e-reader) device, a tablet computer, a smartphone, a smartwatch or similar device. Accordingly, the client device 110 can include computing resources (not shown) comprising, for example, central processing units (CPUs), graphics processing units (GPUs), tensor processing units (TPUs), memory, disk space, incoming bandwidth, and/or outgoing bandwidth, interface(s) (such as I/O interfaces or APIs, or both); controller devices(s); power supplies; a combination of the foregoing; and/or similar resources. The client device 110 can include, or can be functionally coupled to, a display device (not depicted in FIG. 1A).

The client device 110 can be functionally coupled to a pre-operative assessment subsystem 130 by means of one or several networks 120 (wireline network(s), wireless network(s), or a combination thereof). The pre-operative assessment subsystem 130 can include a server application 124 that is retained in one or more memory devices 132. The pre-operative assessment subsystem 130 can be embodied in, or can include, one or multiple server devices. The server application 134 can include software components that can be executed by one or more processors (not depicted in FIG. 1A) integrated into the server device(s). In response to execution, the server application 134 can provide the various functionalities described herein.

The client device 110 can include a web browser or another type of client application (not depicted in FIG. 1A) that permits accessing a uniform resource located (URL) corresponding to a webpage hosted by the server application 134. Some of functionality provided by the server application 134 can be accessed via the webpage. Specifically, accessing that URL can cause the pre-operative assessment subsystem 130 to prompt the subject 104 to self-authenticate using an authentication service. The pre-operative assessment subsystem 130 can be subscribed to the authentication service. In some embodiments, such a service can be an enterprise-grade authentication service. The authentication service can be embodied in an Identity as a Service (IaaS) platform. Multiple authentication service devices 140 constitute, and provide, the authentication service.

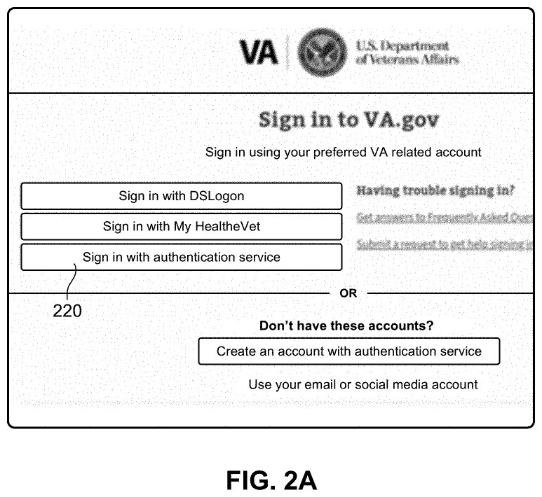

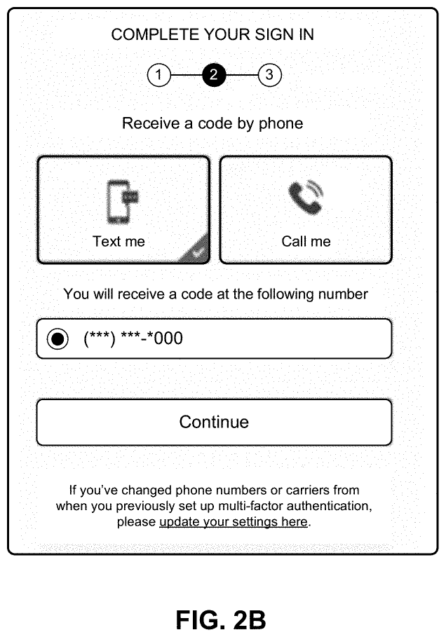

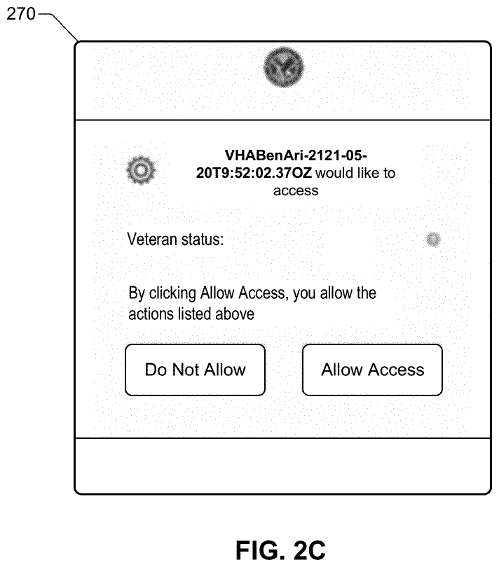

To prompt the subject 104 to self-authenticate, the server application 134 can cause the client device 110 to present a sequence of user interfaces 114. To that, the client device 110 can direct a display device integrated therein to present such a sequence. Some user interfaces in the sequence of user interfaces 114 can be presented in response to defined user-interaction with those user interfaces. More specifically, by navigating to the URL corresponding to the webpage hosted by the server application 134, the client device 110 can direct the display device to present a first user interface of the sequence of user interfaces 114. A non-limiting example of the first user interface is illustrated in the FIG. 2A.

That first user interface can include a selectable visual element (e.g., UI element 220 (FIG. 2A)) that, in response to being selected, causes the server application 134 to direct the client device 110 to the authentication service or a device of the authentication service devices 140. That device can be an authentication server 144, for example. As a result, that device can cause the client device 110 to present a second user interface in the sequence of user interfaces 114. The second user interface can permit the client device 110 to receive input data defining a user identifier (ID). The user ID can correspond to the subject 104 and can be one or a combination of a username, a password, a generated data structure, or an access token. The client device 110 can send, via one or more of the networks 120, the user ID to the device of the authentication service devices 140. In response, in some embodiments, the authentication server 144 can cause the client device 110 to present a third user interface in the sequence of user interfaces 114. That third user interface can permit accessing two-factor authentication functionality. A non-limiting example of the third user interface is illustrated in FIG. 2B.

At least one first device of the authentication service devices 140, such as the authentication server 144, can determine if the user ID satisfies one or multiple access rules. In cases where the two-factor authentication is enabled, the at least one first device or at least one second device of the authentication service devices 140 can validate two-factor data received from the client device 110. In situations where the user ID fails to satisfy an access rule, the at least one first device of the authentication service devices 140, such as the authentication server 144, can cause the pre-operative assessment subsystem 130 to implement an exception process. It is noted that in some cases, the authentication server 144 can implement the exception process.

In the alternative, in situations where the user ID satisfies the access rule(s), the server application 134 can establish a communication session with the client device 110. The authentication service can secure the communication session. The server application 134 also can cause the pre-operative assessment subsystem 130 to present a fourth user interface in the sequence of user interfaces 114. The fourth user interface can prompt configuration of access to a suite of applications (not depicted in FIG. 1A) that can be used via the client device 110. A non-limiting example of the fourth user interface is illustrated in FIG. 2C.

In addition, or in other embodiments, when the user ID satisfies the access rule(s), the authentication server 144 and/or another device of the authentication service devices 140 can authenticate a user account of the subject 104 and can redirect the client device 110 to the pre-operative assessment subsystem 130.

FIG. 1B schematically summarizes an example of data flow involved in authentication and access to a secure computer-based pre-operative assessment, as is described herein, in accordance with one or more embodiments of the disclosure. As is illustrated in FIG. 1B, as part of the data flow, the client device 110 can send a request for access to the server application 134. In response to receiving the request, the server application 134 can redirect the client device 110 to the authentication server 144, for example. The authentication server 144 can, in turn, redirect the client device 110 to a login page. The client device 110 can then provide credentials (e.g., username and password) to the authentication server 144. In some cases, based on the credentials, the authentication server 144 can authenticate a user account pertaining to the subject 104. The authentication server 144 can then redirect the client device 110 to the server application 134 after such an authentication.

As a result authenticating that user account, the pre-operative assessment subsystem 130 can cause the client device 110 to output of a graphical user interface (GUI) 118 configured to elicit one or multiple responses. For example, the GUI 118 can include one or multiple prompts (such as questions) represented by textual elements or visual elements, or a combination of both. Causing output of the GUI 118 can include causing presentation of the GUI 118 at the client device 110. To cause presentation of the GUI 118 at the client device 110, the pre-operative assessment subsystem 130 can cause the client device 110 to direct a display device to present the GUI 118. The display device can be integrated into the client device 110 or functionally coupled thereto. At least one of the response(s) can be associated with a user condition. In some cases, the user condition can be one or a combination of a pre-operative condition, a post-operative condition, a mental health condition, a wellness state, or a disease state.

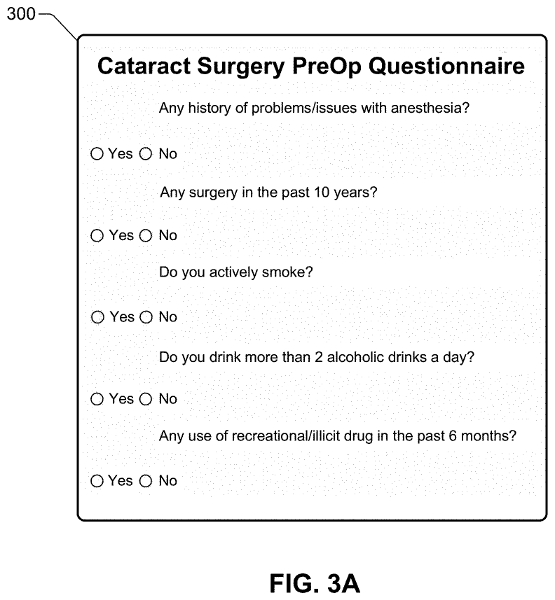

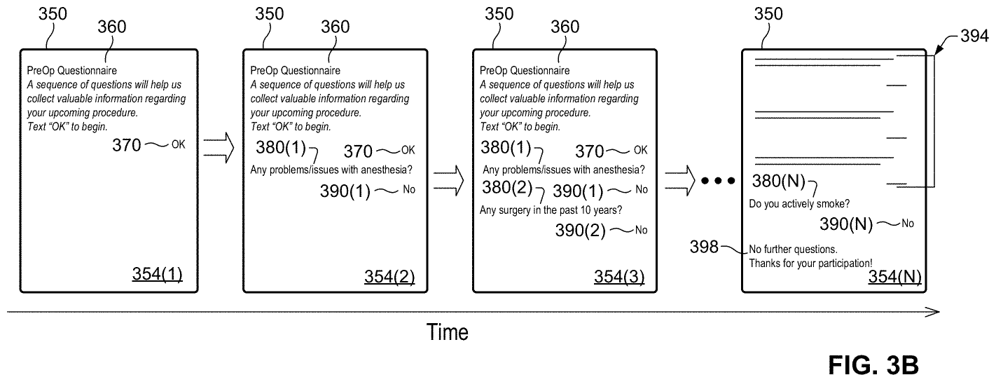

Accordingly, as part of a pre-operative protocol, the GUI 118 can be configured to elicit the one or multiples responses by presenting one or more questions (or, in some configurations, queries, or other types of prompts) associated with the user condition. Thus, the GUI 118 can include several UI elements (selectable and non-selectable, for example) and/or other digital content that conveys the question(s). Embodiments of this disclosure can be applied to many surgical procedures, so the content of the GUI 118 can be specific to a surgical procedure. In some embodiments, the GUI 118 can convey a questionnaire or another type of assessment associated with a forthcoming surgery, such as cataract surgery. The GUI 300 illustrated in FIG. 3A is a non-limiting example of the GUI 118. The client device 110 can receive input data from the subject 104 corresponding to the user ID that has been authenticated, the input data define the one or multiple responses elicited by the GUI 118.

The disclosure is not limited to presenting questions (or, in some configurations, queries) in a GUI, such as the GUI 118. In some embodiments, the pre-operative assessment subsystem 130 can implement a text bot, or another type of software module, that permit the exchange of information with the client device 110 by exchanging electronic messages. The electronic messages can be exchanged in response to executing program code that permits receiving and sending electronic messages. The program code can embody a component of the operating system (O/S) of the client device. Examples of electronic messages include short message service (SMS) messages, multimedia message service (MMS) messages, or iMessages. Implementation of the text bot, or that other software module, can cause a display device of the client device 110 to present a sequence of electronic messages that prompt respective responses. The sequence of electronic messages can embody the assessment associated with a foregoing surgery. The respective responses prompted by that sequence can be individually received at the client device 110. In response, the client device 110 can convey to the pre-operative assessment subsystem 130 as response electronic messages.

Simply for purposes of illustration, FIG. 3B presents examples of UIs including textual elements that embody a sequence of prompt electronic messages and another sequence of response electronic messages. Each one of those sequences can be presented in a display device 350 integrated into the client device 110, for example. Prompt electronic messages and response electronic messages are presented alternatingly. Specifically, a UI 354(1) can include a prompt electronic message 360 that presents a description of the purpose of the exchange of electronic messages and prompt for continuing the sequence of prompt electronic messages. The UI 354(1) also includes a response electronic message 370 that can cause the sequence of prompt electronic messages to proceed.

The client device 110 can present, via the display device 350, a UI 354(2) in response to the response electronic message 370. The UI 354(2) includes a prompt electronic message 380(1) conveying a question pertaining to a pre-operative assessment associated with a foregoing surgery. The client device 110 can receive input information defining a response electronic message 390(1) conveying an answer to that question. The UI 354(2) also includes the response electronic message 390(1). The client device 110 can send data identifying the answer to the pre-operative assessment subsystem 130 (FIG. 1A).

The sequence of prompt electronic messages can continue. To that end, the client device 110 can present, via the display device 350, a UI 354(3) in response to the response electronic message 390(1). The UI 354(3) can include a prompt electronic message 380(2) conveying another question pertaining to the pre-operative assessment. The client device 110 can receive input information defining a response electronic message 390(2) conveying an answer to that question. The UI 354(2) also includes the response electronic message 390(2). The client device 110 can send data identifying the answer to the pre-operative assessment subsystem 130 (FIG. 1A).

Sequences of alternating prompt electronic messages and response electronic messages can continue. A terminal portion of those sequences is depicted as electronic messages 394. The sequences can be presented until the display device presents a UI 354(N) that includes a prompt electronic message 380(N) conveying a terminal question pertaining to the pre-operative assessment. That is the pre-operative assessment can have N questions. The client device 110 can receive input information defining a response electronic message 390(N) conveying a terminal answer to the terminal question. The UI 354(N) also includes the response electronic message 390(N). The client device 110 can send data identifying the answer to the pre-operative assessment subsystem 130 (FIG. 1A). The client device 110, via the display device 350, can present a closing message 398 within the UI 354(N).

Regardless of the manner of collecting the one or multiple responses, the server application 134 can receive, via at least one of the network(s) 120, input data defining the one or multiple responses. The input data can be received in separate transmissions or in a single transmission. In response, the server application 134 can retain the received input data in a secure HIPAA compliant database 136 (referred to as assessment data 136) managed by a secure server device (not depicted) included in the pre-operative assessment subsystem 130. The server application 134 can supply the input data to one or multiple other applications in several formats, including, for example, industry standards for clinical data transfer. Such standards can include, for example, Fast Health Interoperability Resource (FHIR) and JavaScript Object Notation (JSON). The subject 104 also can be logged out and the session information can be eliminated. In further response, the server application 134 can cause the pre-operative assessment subsystem 130 to associate the one or multiple responses with the user ID. The pre-operative assessment subsystem 130 can associate the one or multiple responses with the user ID by at least generating a data structure including a representation (e.g., a data record or metadata) of each response of the one or multiple responses and a key value corresponding to the user ID. The representation of each response of the one or multiple responses can be embodied in, or can include, an encoded value. The key value can be a numerical value or an alphanumerical code.

The server application 134 also can cause the pre-operative assessment subsystem 130 to generate, using the one or multiple responses, a visual representation, an aural representation, and/or a somatosensory representation. Those representations, individually or in combination, can be indicative of the user condition. A non-limiting example of the somatosensory representation is a haptic representation that can cause a device (a user device or a client device, for example) to convey the user condition by means of motion or the application of pressure. The visual representation can include graphical elements (a still image or an animation, for example) or textual elements, or a combination of graphical elements and textual elements. In some embodiments, the pre-operative assessment subsystem 130 can generate the visual representation by at least determining a graphical layout of the graphical and/or textual elements based on the number of the one or multiple responses and also based on the respective representations of each response of the one or multiple responses. That graphical layout can include one or more of (i) a UI object associated with each representation of each response of the one or multiple responses; (ii) a position of the UI object within a viewport encompassing the visual representation; (iii) time of the UI object, or (iv) a color of the object.

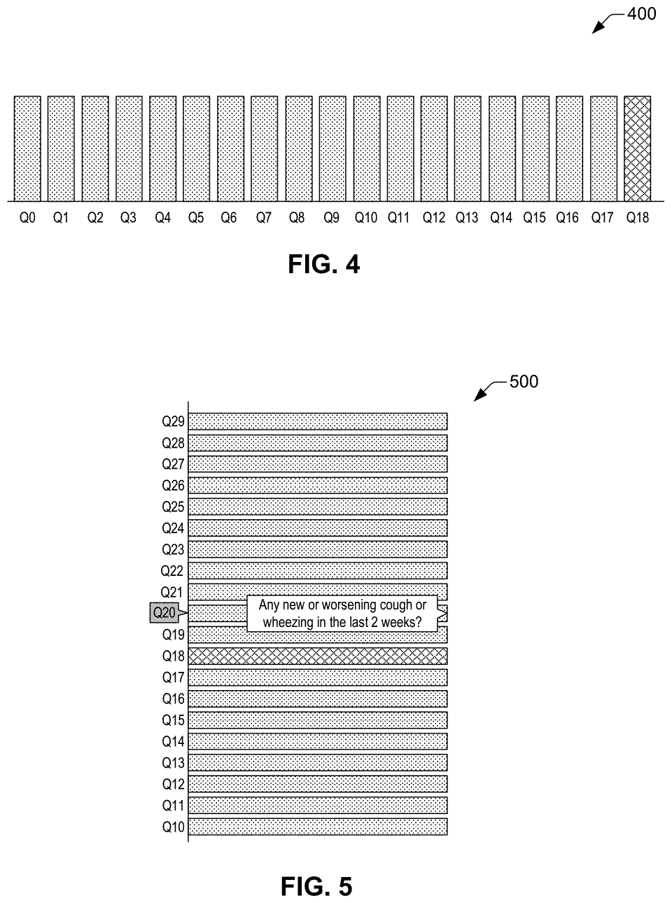

Because a visual representation indicates the user condition in connection with a surgical procedure, for example, the pre-operative assessment subsystem 130, via the server application 134, can generate one or more elements (graphical or textual) of the visual representation to reveal relative importance of two or more responses that characterize the user condition. As a non-limiting example, a first response to a survey (such as a pre-operative assessment or questionnaire) can be represented visually by a rectangle having a cool color (e.g., blue or green) or a non-conspicuous type of markings (e.g., sparse stippling), representing that the first response does not create an issue related to the surgical procedure. In turn, a second response to the survey can be represented visually by another rectangle having a hot color (e.g., red or yellow) or a conspicuous type of markings (e.g., dense stippling or dense cross-hatching), representing that the second response potentially creates an issue related to the surgical procedure. Accordingly, such a visual representation can convey actionable information at a glance. Thus, an end-user (e.g., a healthcare provider) that reviews the responses to a survey can determine, based on the type of visual representation of a response, if a response may require further inquiry or is in agreement with moving forward with an operative procedure without further inquiry. The visual representation 400 shown in FIG. 4 and the visual representation 500 shown in FIG. 5 are non-limiting examples of visual representations of multiple responses to a survey or assessment in accordance with this disclosure. Each one of the visual representation 400 and the visual representation 500 convey actionable information at a glance. Specifically, in the visual representation 400, respective responses to questions Q0, Q2, continuing up to question Q17 are represented visually in a manner indicative of agreement with moving forward with an operative procedure without further review. In turn, the response to question Q18 is represented visually in a manner indicative of potential need for further review. In the visual representation 500, respective responses to questions Q0 to Q17 and questions Q19 to Q29 are represented visually in a manner indicative of agreement with moving forward with an operative procedure without further review. In turn, the response to question Q18 is represented visually in a manner indicative of potential need for further review. In some cases, visual elements corresponding to respective questions within a visual representation can be selectable or otherwise interactive. As such, in response to a click, tap, swipe or another gesture (such as hovering over a selectable visual element), or yet another type of interaction, the client device 150 can redraw the visual representation 500 to present a question or prompt corresponding to the visual element being selected. Simply as an illustration, in the visual representation 500, the question Q20 is shown as an overlay in response to the visual element corresponding to Q20 being selected. In some cases, questions Q10 to Q29 represented in FIG. 5 can be part of the same survey that includes questions Q1 to Q18 represented in FIG. 4.

Thus, visual representations and aural representations of this disclosure can provide an intuitive and easy to understand characterization of a user condition related to a forthcoming procedure. As a result, the visual representations can simplify decision-making processes for a practicing clinician involved in that procedure.

In some embodiments, the server application 134 can include one or more components that provide functionality accessible to clinical staff. At least one of those component(s) can access assessment data for a subject and a visual representation of that assessment data, and can cause the pre-operative assessment subsystem 130 to supply the visual representation to a client device 150. That assessment data can be contained in assessment data 136. Those component(s) also can cause the pre-operative assessment subsystem 130 to supply an aural representation corresponding to the assessment data to the client device 150. The client device 110 can be embodied in, for example, a personal computer, a laptop computer, an electronic-reader (e-reader) device, a tablet computer, a smartphone, a smartwatch or similar device. Accordingly, the client device 150 can include computing resources (not shown) comprising, for example, central processing units (CPUs), graphics processing units (GPUs), tensor processing units (TPUs), memory, disk space, incoming bandwidth, and/or outgoing bandwidth, interface(s) (such as I/O interfaces or APIs, or both); controller devices(s); power supplies; a combination of the foregoing; and/or similar resources. The client device 150 can include, or can be functionally coupled to, a display device (not depicted in FIG. 1A).

In some embodiments, supplying the visual representation includes causing the client device 150 to output the visual representation. For instance, the pre-operative assessment subsystem 130 can cause the client device 150 to direct a display device to present a user interface 154 according to the visual representation. The display device can be integrated into the client device 150 or functionally coupled thereto. In addition, or in other embodiments, supplying an aural representation includes causing output of the aural representation at the client device 150. For instance, the pre-operative assessment subsystem 130 can cause the client device 150 to direct an audio output unit (a speaker or a haptic device, for example) to present the aural representation. Further, or in yet other embodiments, rather than causing the client device 150 to present a visual representation or aural representation, the pre-operative subsystem 130 can cause the client device 150 can present a somatosensory representation of (i) one or more responses and/or (ii) a condition of the subject 104.

The server application 134 can cause the pre-operative assessment subsystem 130, or a component thereof, to supply a visual representation and/or an aural representation in response to receiving a query message that includes a request to access the one or multiple responses associated with an assessment of a user condition of a subject (e.g., subject 104).

In some embodiments, data indicative of responses to a pre-operative survey or other types of questionnaires (such as an electronic clinical outcome assessment (eCOA) or a neuropsychological assessment) can be obtained in other ways. In some cases, with reference to FIG. 1C, the client device 110 can present, after a survey or questionnaire has been completed, a selectable visual element indicative of a prompt to receive a token. In one example, the token is a QR code or a barcode. In another example, the toke is a non-fungible token (NFT). In response to receiving input data indicative of the token being desired, the client device 110 can present a second prompt to select the manner of receiving the token. For example, the second prompt can request the subject 104 to select one of several forms of electronic communication to receive the token. For example, the token can be received via email or electronic messaging (e.g., SMS, MIMS, iMessage). As such, the second prompt can permit entering an email address or a mobile telephone number. In further response, the client device can send a request message for the token to the pre-operative assessment subsystem 130, where the request message can include payload data indicative of the electronic address (e.g., email address or mobile telephone number) desired for communication of the token. One or more components present in the pre-operative assessment subsystem 130 can generate the token and can send the token to the client device 110. Generating the token can include creating an address (e.g., a uniform resource locator (URL)) where the data indicative of the responses is retained within a network of computing devices. In one example, the address can be indicative of the data storage 132 where the responses are retained as part of assessment data 136. Sending the token includes sending data defining the token to the electronic address indicated in the request message. The data defining the token include first data indicative of the address where the data indicative of the responses is stored. In some cases, such data can include formatting information that can permit the client device 110 to draw a visual representation of the token in a UI. The client device 110 can draw that visual representation via a UI library (e.g., a UI toolkit) therein and a messaging application (note depicted in FIG. 1C) included in the client device, for example.

The client device 110 can then move to a location proximate to the client device 150. Such movement represent by an dash-line arrow in FIG. 1C. The client device 110 can then be caused to present a visual representation of the token, or the toke itself in cases the token is a QR code. For example, causing presentation of the visual representation of the token can include executing a messaging application (e.g., an email application) within the client device 110 and presenting a UI containing email content including the token or the visual representation thereof.

The client device 150 can be functionally coupled to a reader device 160 that can optically scan (or otherwise capture) a token or a visual representation 170 of the token. In response, the client device 150 can obtain data of the address (e.g., a URL) where the data indicative of the responses to the pre-operative survey or questionnaire is stored. The client device 150, using that address, can access such data and can present the responses in the UI 154 as is described herein.

By providing a token, embodiments of this disclosure can permit efficiently accessing responses to pre-operative surveys and/or other types of questionnaires. Such efficient access can mitigate or entirely avoid human intervention to access such responses.

Non-limiting Example Scenario—Cataract Surgery Scenario. Cataract Surgery is a common low risk procedure done on older patients in their 7th, 8th and 9th decades of life. This procedure is accomplished successfully in the majority of patients using minimal sedation and local anesthetics applied by the operating ophthalmologist. A minimal requirement is that the patient can lie still for the duration of the procedure allowing the surgeon to operate.

It is the standard of practice to offer pre-operative anesthetic evaluation to these patients on the account of the medical complexity they often present. By far and large while medically complex, patients successfully undergo the procedure with minimal anesthetic intervention.

On rare occasions, a patient cannot tolerate or cannot cooperate for the duration of surgery while presenting an anesthetic challenge to the anesthesiologist taking care of the patient. In other words, anesthesiologists are presented with two extreme scenarios: (i) the majority of cases that can safely be done with minimal sedation and (ii) the rare event where anesthetic management becomes extremely complex necessitating further evaluation and more decision making.

The low expectation of challenges calls into question whether a full pre-operative evaluation is indeed worthwhile the anesthesiologist's time investment. As such the majority of such consultations are phone consultations or chart reviews. However, not interviewing or examining the patient puts the anesthesiologist at a disadvantage to address pertinent questions that may or may not be available in the chart necessitating a large time investment to study the chart, in what is otherwise a process with low expectation of yielding anything meaningful.

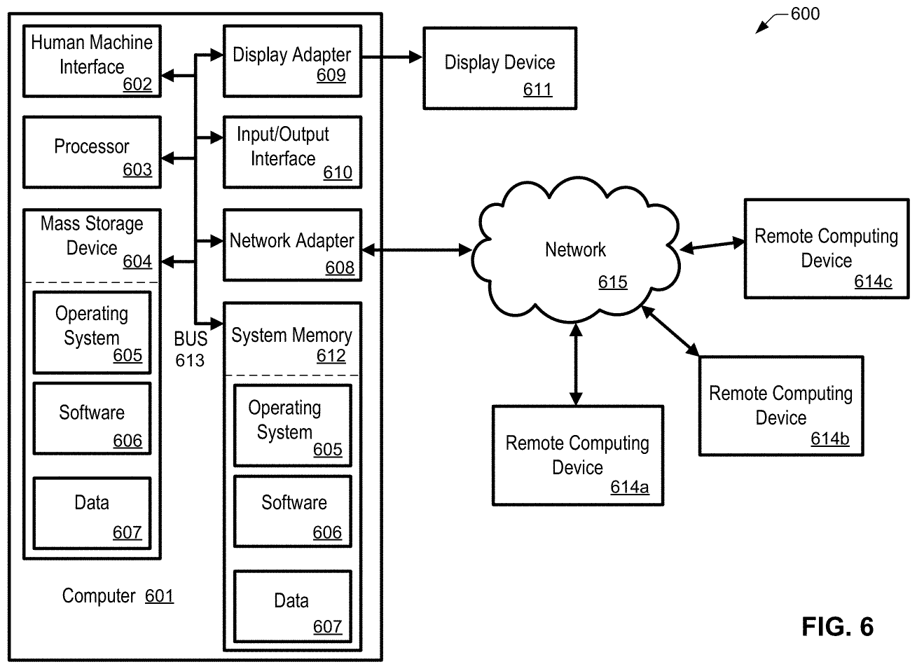

In order to provide some context, the computing systems, computing devices, computer-program products, and techniques of this disclosure can be implemented on a computer 601 as illustrated in FIG. 6 and described below. Similarly, the computing systems, computing devices, computer-program products, and techniques disclosed herein can utilize one or more computers, or computing devices, to perform one or more functions in one or more locations. FIG. 6 is a block diagram illustrating an example computing system 600 for performing the disclosed methods. This example computing system 600 is only an example of a computing system and is not intended to suggest any limitation as to the scope of use or functionality of computing system architecture. Neither should the computing system 600 be interpreted as having any dependency or requirement relating to any one or combination of components illustrated in the example computing system. In some embodiments, the example computing system can embody, or can include, the computing system 100 (FIG. 1A and FIG. 1C).

The present methods and systems can be operational with numerous other general purpose or special purpose computing system environments or configurations. Non-limiting examples of well-known computing systems, environments, and/or configurations that can be suitable for use with the systems and methods comprise, but are not limited to, personal computers, server computers, laptop devices, and multiprocessor systems. Additional non-limiting examples comprise set-top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods disclosed herein can be implemented via a general-purpose computing device in the form of a computer 601. The components of the computer 601 can comprise, but are not limited to, one or more processors 603, a system memory 612, and a system bus 613 that couples various system components including the one or more processors 603 to the system memory 612. The system can utilize parallel computing.

The system bus 613 represents one or more of several possible types of bus structures, including a memory bus or memory controller, a peripheral bus, an accelerated graphics port, or local bus using any of a variety of bus architectures. The bus 613, and all buses specified in this description can also be implemented over a wired or wireless network connection and each of the subsystems, including the one or more processors 603, a mass storage device 604, an operating system 605, software 606, data 607, a network adapter 608, the system memory 612, an Input/Output Interface 610, a display adapter 609, a display device 611, and a human-machine interface 602, can be contained within one or more remote computing devices 614a, b, c at physically separate locations, connected through buses of this form, in effect implementing a fully distributed system.

The computer 601 typically comprises a variety of computer-readable media. Exemplary readable media can be any available media that is accessible by the computer 601 and comprises, for example and not meant to be limiting, both volatile and non-volatile media, removable and non-removable media. The system memory 612 comprises computer readable media in the form of volatile memory, such as random access memory (RAM), and/or non-volatile memory, such as read only memory (ROM). The system memory 612 typically contains data such as the data 607 and/or program modules such as the operating system 605 and the software 606 that are immediately accessible to and/or are presently operated on by the one or more processors 603.

In another aspect, the computer 601 can also comprise other removable/non-removable, volatile/non-volatile computer storage media. By way of example and not limitation, FIG. 6 illustrates the mass storage device 604 which can provide non-volatile storage of computer code, computer readable instructions, data structures, program modules, and other data for the computer 601. For example and not meant to be limiting, the mass storage device 604 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 604, including by way of example and not limitation, the operating system 605 and the software 606. Each of the operating system 605 and the software 606 (or some combination thereof) can comprise elements of the programming and the software 606. The data 607 can also be stored on the mass storage device 604. The data 607 can be stored in any of one or more databases known in the art. Non-limiting examples of such databases comprise, DB2®, Microsoft® Access, Microsoft® SQL Server, Oracle®, mySQL, PostgreSQL, and the like. The databases can be centralized or distributed across multiple systems. The data 607 can include, among other data, the assessment data 136.

In an aspect, the software 606 can comprise various processor-executable components that provide at least some of the functionality of the computer 601. In an aspect, the software 606 can comprise the processor-executable image of the server application 134 (FIG. 1A) and/or an interface to that processor-executable image of the server application 134 (FIG. 1A).

In another aspect, the user can enter commands and information into the computer 601 via an input device (not shown). Non-limiting examples of such input devices comprise, but are not limited to, a keyboard, pointing device (e.g., a “mouse”), a microphone, a joystick, a scanner, tactile input devices such as gloves, and other body coverings, and the like. These and other input devices can be connected to the one or more processors 603 via the human-machine interface 602 that is coupled to the system bus 613, but can be connected by other interface and bus structures, such as a parallel port, game port, an IEEE 1394 Port (also known as a Firewire port), a serial port, or a universal serial bus (USB).

In yet another aspect, the display device 611 can also be connected to the system bus 613 via an interface, such as the display adapter 609. It is contemplated that the computer 601 can have more than one display adapter 609 and the computer 601 can have more than one display device 611. As a non-limiting example, the display device 611 can be a monitor, an LCD (Liquid Crystal Display), or a projector. In addition to the display device 611, other output peripheral devices can comprise components such as speakers (not shown) and a printer (not shown) which can be connected to the computer 601 via the Input/Output Interface 610. Any operation and/or result of the methods of this disclosure can be output in any form to an output device. Such output can be any form of visual representation, including, but not limited to, textual, graphical, animation, audio, tactile, and the like. The display device 611 and computer 601 can be part of one device, or separate devices.

The computer 601 can operate in a networked environment using logical connections to one or more remote computing devices 614a, b, c. By way of example and not limitation, a remote computing device can be a personal computer, portable computer, smartphone, a server, a router, a network computer, a peer device or other common network node, and so on. Logical connections between the computer 601 and a remote computing device 614a, b, c can be made via one or more networks 615 (generically referred to as network 615), such as a local area network (LAN) and/or a general wide area network (WAN). The network 615 can embody the network(s) 120. Such network connections can be through the network adapter 608. The network adapter 608 can be implemented in both wired and wireless environments. In an aspect, one or more of the remote computing devices 614a, b, c can comprise an external engine and/or an interface to the external engine. While not illustrated, at least one of the remote computing devices 614a, b, c can include respective display devices or can be functionally coupled to respective display devices. In some embodiments, the computer 601 can embody the pre-operative assessment subsystem 130, a first computing device of the remote computing devices 614a, b, c can embody the client device 110, and a second computing device of the remote computing devices 614a, b, c can embody the client device 150.

For purposes of illustration, application programs and other executable program components such as the operating system 605 are illustrated herein as discrete blocks, although it is recognized that such programs and components reside at various times in different storage components of the computing device 601, and are executed by the one or more processors 603 of the computer. An implementation of the software 606 can be stored on or transmitted across some form of computer-readable media. Any of the disclosed methods can be performed by computer readable instructions embodied on computer-readable media. Computer-readable media can be any available media that can be accessed by a computer. By way of example and not meant to be limiting, computer-readable media can comprise “computer storage media” and “communications media.” “Computer storage media” comprise volatile and non-volatile, removable and non-removable media implemented in any methods or technology for storage of information such as computer-readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

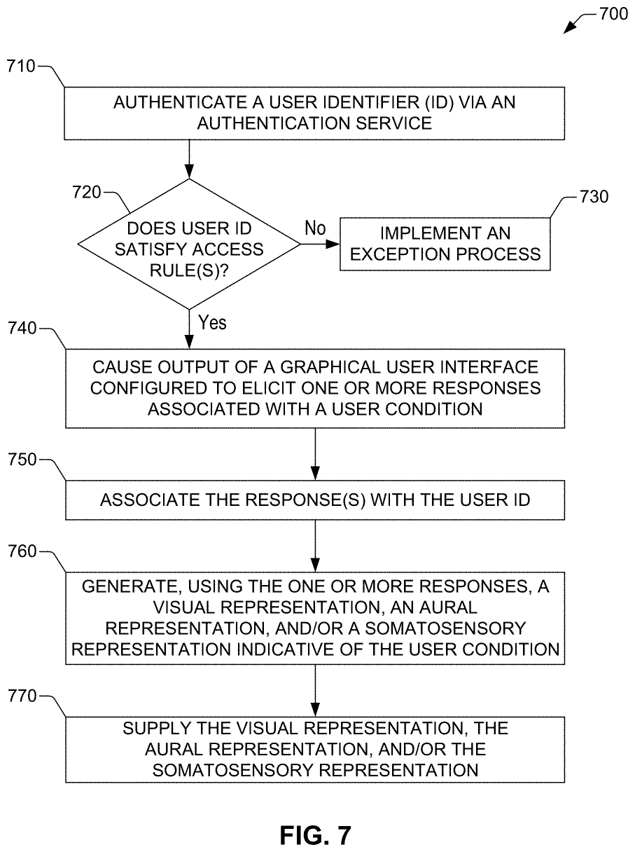

In view of the various aspects of the techniques disclosed herein, a non-limiting example method that can be implemented in accordance with embodiments of this disclosure can be more readily appreciated with reference to the flowchart in FIG. 7. For purposes of simplicity of explanation, the non-limiting example methods disclosed herein are presented and described as a series of blocks (with each block representing an action or an operation in a method, for example). However, it is to be understood and appreciated that the disclosed methods are not limited by the order of blocks and associated actions or operations, as some blocks may occur in different orders and/or concurrently with other blocks from that are shown and described herein. As a non-limiting example, the various methods or processes of the disclosure can be alternatively represented as a series of interrelated states or events, such as in a state diagram. Furthermore, not all illustrated blocks, and associated action(s), may be required to implement a method in accordance with one or more aspects of the disclosure. Further yet, two or more of the disclosed methods or processes can be implemented in combination with each other, to accomplish one or more functionalities and/or advantages described herein.

The methods of the disclosure can be retained on an article of manufacture, or computer-readable non-transitory storage medium, to permit or facilitate transporting and transferring such methods to a computing device for execution, and thus implementation, by a processor of the computing device or for storage in a memory thereof or functionally coupled thereto. Such a computing device can be embodied in a mobile computer, such as an electronic book reader (e-reader) or other tablet computers, or a smartphone; a mobile gaming console; or the like. In one aspect, one or more processors, such as processor(s) that implement one or more of the disclosed methods, can be employed to execute program instructions retained in a memory, or any computer- or machine-readable medium, to implement the one or more methods. The program instructions can provide a computer-executable or machine-executable framework to implement the methods described herein.

FIG. 7 illustrates a non-limiting example of a method 700 for a secure computer-based pre-operative assessment, in accordance with one or more embodiments of the disclosure. A computing system can implement, entirely or partially, the non-limiting example method 700. To that end, the computing system includes computing resources that can implement at least one of the blocks included in the non-limiting example method 700. The computing resources include, for example, central processing units (CPUs), graphics processing units (GPUs), tensor processing units (TPUs), memory, disk space, incoming bandwidth, and/or outgoing bandwidth, interface(s) (such as I/O interfaces); controller devices(s); power supplies; and the like. For instance, the memory can include programming interface(s) (such as APIs); an operating system; software for configuration and or control of a virtualized environment; firmware; and similar resources.

In some embodiments, the computing system can embody, or can constitute, the pre-operative assessment subsystem 130. In other embodiments, the computing system can embody, or can include, the computing system 100 (FIG. 1). As is described herein, in some cases, the example computing system 601 (FIG. 6) can embody, or can include, the computing system that implements the example method 700. The computing system that implements that example method 700 can include one or more computing devices that host the server application 134, and can implement one or more of blocks of the example method 700 in response to execution of the server application 134. At least one processor of such computing device(s) can execute the server application 134.

At block 710, the computing system can authenticate a user identifier (ID) via an authentication service. As mentioned, in some cases, the authentication service can be embodied in, or can include, an Identity as a Service (IaaS) platform. The user ID corresponds to a subject and can be one or a combination of a user name, a password, a generated data structure, or an access token.

At block 720, the computing system can determine if the user ID satisfies one or multiple access rules. In response to a negative determination, the computing system can implement an exception process at block 730.

In response to an affirmative determination, the flow of the non-limiting example method 700 can continue to block 740, at which block the computing system can cause output of a graphical user interface (GUI) configured to elicit one or multiple responses. At least one of the response(s) can be associated with a user condition. In some cases, the user condition can be one or a combination of a pre-operative condition, a post-operative condition, a mental health condition, a wellness state, or a disease state. Causing output of the GUI can include causing presentation of the GUI at a client device (e.g., client device 110 (FIG. 1)). The client device can receive input data from the subject corresponding to the user ID that has been authenticated, the input data defining the response(s).

In some cases, the GUI is configured to elicit the one or multiples responses associated with the user condition by presenting one or more questions (or, in some configurations, queries) associated with the user condition. In some embodiments, the GUI can be a questionnaire or another type of assessment associated with a forthcoming surgery. See GUI 300 (FIG. 3A), for example.

In addition, or instead of causing output of the GUI, the computing system can cause presentation of a sequence of alternating and adapting prompt electronic messages and response electronic messages, at block 740. See FIG. 3B and related description, for example. The prompt electronic messages can be adaptive based on one or multiple algorithms to generate a natural language (NL) statement that is responsive to another NL statement (e.g., a response electronic message) and is substantially logically sound. The algorithm(s) can include decision support trees, for example.

At block 750, the computing system can associate the one or multiple responses with the user ID. Associating the one or multiple responses with the user ID can include generating a data structure including a representation (e.g., a data record or metadata) of each response of the one or multiple responses and a key value corresponding to the user ID. The representation of each response of the one or more responses can be embodied in, or can include, an encoded value.

At block 760, the computing system can generate, using the one or multiple responses, a visual representation, an aural representation, a somatosensory representation, or a combination of the foregoing. Those representations, individually or in combination, can be indicative of the user condition. In some embodiments, generating the visual representation includes determining a graphical layout of the visual representation based on the number of the one or multiple responses and also based on the respective representations of each response of the one or multiple responses. That graphical layout can include one or more of an object associated with each representation of each response of the one or multiple responses, a position of the object, time of the object, or a color of the object.

At block 770, the computing system can supply at least one of the visual representation, the aural representation, or the somatosensory representation generated at block 760. In cases where only the visual representation has been created, the computing system can supply that visual representation. In cases where only the aural representation has been created, the computing system can supply that aural representation. In cases where both the visual representation and the aural representation have been created, the computing system can supply the visual representation and the aural representation. Supplying the visual representation or the aural representation, or both, can be responsive to receiving a query message and can include causing output of the visual representation or the aural representation, or both. The query message can include a request to access the one or multiple responses associated with the user condition.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. When values are expressed as approximations, by use of the antecedent “about,” it will be understood that the particular value forms another configuration. It will be further understood that the endpoints of each of the ranges are significant both in relation to the other endpoint, and independently of the other endpoint.

Throughout the description and claims of this specification, the words “include” and “comprise” and variations of the word, such as “including,” “comprising,” “includes” and “comprises,” mean “including but not limited to,” and are not intended to exclude other components, integers or steps. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memristors, Non-Volatile Random Access Memory (NVRAM), flash memory, or a combination thereof.

Embodiments of this disclosure have been described with reference to diagrams, flowcharts, and other illustrations of computer-implemented methods, systems, apparatuses, and computer program products. Each block of the block diagrams and flowchart illustrations, and combinations of blocks in the block diagrams and flowchart illustrations, respectively, may be implemented by processor-accessible instructions. Such instructions may include, for example, computer program instructions (e.g., processor-readable and/or processor-executable instructions). The processor-accessible instructions may be built (e.g., linked and compiled) and retained in processor-executable form in one or multiple memory devices or one or many other processor-accessible non-transitory storage media. These computer program instructions (built or otherwise) may be loaded onto a general-purpose computer, special purpose computer, or other programmable data processing apparatus to produce a machine. The loaded computer program instructions may be accessed and executed by one or multiple processors or other types of processing circuitry. In response to execution, the loaded computer program instructions provide the functionality described in connection with flowchart blocks (individually or in a particular combination) or blocks in block diagrams (individually or in a particular combination). Thus, such instructions which execute on the computer or other programmable data processing apparatus create a means for implementing the functions specified in the flowchart blocks (individually or in a particular combination) or blocks in block diagrams (individually or in a particular combination).

These computer program instructions may also be stored in a computer-readable memory that may direct a computer or other programmable data processing apparatus to function in a particular manner, such that the instructions stored in the computer-readable memory produce an article of manufacture including processor-accessible instruction (e.g., processor-readable instructions and/or processor-executable instructions) to implement the function specified in the flowchart blocks (individually or in a particular combination) or blocks in block diagrams (individually or in a particular combination). The computer program instructions (built or otherwise) may also be loaded onto a computer or other programmable data processing apparatus to cause a series of operations to be performed on the computer or other programmable apparatus to produce a computer-implemented process. The series of operations may be performed in response to execution by one or more processor or other types of processing circuitry. Thus, such instructions that execute on the computer or other programmable apparatus provide operations for implementing the functions specified in the flowchart blocks (individually or in a particular combination) or blocks in block diagrams (individually or in a particular combination).

Accordingly, blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

As is used in this specification and annexed drawings, the terms “module,” “component,” “system,” “platform,” and the like, can refer to and/or can include a computer-related entity or an entity related to an operational machine with one or more specific functionalities. Such entities can be either hardware, a combination of hardware and software, software (program code or executable program code, for example), or software in execution. In one example, a component can be a process running on a processor, a processor, an object, an executable (e.g., binary software), a thread of execution, a computer program, and/or a computing device. Simply as an illustration, a software application running on a server device can be a component and the server device also can be a component. One or more modules can reside within a process and/or thread of execution. One or more components also can reside within a process and/or thread of execution. Each one of a module and a component can be localized on one computing device and/or distributed between two or more computing devices. In another example, respective components (or modules) can execute from various computer-readable storage media having various data structures stored thereon. The components (or modules) can communicate via local and/or remote processes such as in accordance with a signal having one or more data packets (e.g., data from one component interacting with another component in a local system, distributed system, and/or across a network such as the Internet with other systems via the signal). As another illustrations, in some cases, a component can emulate an electronic component via a virtual machine, e.g., within a cloud computing system. The terms “module” and “component” (and their plural versions) may be used interchangeably where clear from context, in some cases.

As is used in this specification and annexed drawings, the term “processor” can refer to substantially any computing processing unit or computing device, including single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to electronic circuitry designed in assembled to execute code instructions and/or operate on data and signaling. Such electronic circuitry can be assembled in a chipset, for example. Accordingly, in some cases, a processor can be embodied, or can include, an application specific integrated circuit (ASIC), a digital signal processor (DSP), a field programmable gate array (FPGA), a complex programmable logic device (CPLD), a discrete gate or transistor logic, discrete hardware components, or any combination thereof designed and assembled to perform the functionality described herein. Further, in some cases, processors can exploit nano-scale architectures, such as molecular and quantum-dot based transistors, switches and gates, in order to optimize space usage or enhance performance of computing devices. A processor can also be implemented as a combination of computing processing units.

Further, in this specification and annexed drawings, terms such as “storage,” “data storage,” “repository,” and substantially any other information storage component relevant to operation and functionality of a system, subsystem, module, and component are utilized to refer to “memory components,” entities embodied in a “memory,” or components including a memory. As is described herein, memory and/or memory components of this disclosure can be either volatile memory or nonvolatile memory, or can include both volatile and nonvolatile memory. Simply as an illustration, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Embodiments of this disclosure are not limited to these types of memory, and other types of memory devices can be contemplated.

This detailed description may refer to a given entity performing some action. It should be understood that this language may in some cases mean that a system (e.g., a computer or multiple computers) owned and/or controlled by the given entity is actually performing the action.

While the computer-implemented methods, apparatuses, devices, and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

What is claimed is:

1. A computer-implemented method comprising:
authenticating a user identifier via an authentication service;
based on authenticating the user identifier, causing output of a graphical user interface configured to elicit one or more responses associated with a user condition;
receiving the one or more responses via the graphical user interface;
associating the one or more responses with the user identifier;
generating, based on the one or more responses, at least one of a visual representation or an aural representation indicative of the user condition; and
causing output of at least one of the visual representation or the aural representation.

2. The computer-implemented method of claim 1, wherein the authentication service comprises an Identity as a Service (IaaS) platform.

3. The computer-implemented method of claim 1, wherein the user identifier is one or more of a username, a password, a generated data structure, or an access token.

4. The computer-implemented method of claim 1, wherein the user condition is one or more of a pre-operative condition, a post-operative condition, a mental health condition, a wellness screening, or a disease state.

5. The computer-implemented method of claim 1, wherein the graphical user interface is configured to elicit one or more responses associated with a user condition by presenting one or more queries associated with the user condition.

6. The computer-implemented method of claim 1, wherein associating the one or more responses with the user identifier comprises generating a data structure comprising a representation of each response of the one or more responses and a key value corresponding to the user identifier.

7. The computer-implemented method of claim 6, wherein the representation of each response of the one or more responses is an encoded value.

8. The computer-implemented method of claim 6, wherein the generating comprises determining, based on a quantity of the one or more responses and based on the representations of each response of the one or more responses, a graphical layout of the visual representation.

9. The computer-implemented method of claim 8, wherein the graphical layout comprises one or more of an object associated with each representation of each response of the one or more responses, a position of the object, time of the object, or a color of the object.

10. The computer-implemented method of claim 1, further comprising:
receiving a request to access the one or more responses associated with the user condition; and
wherein causing output of the at least one of the visual representation or the aural representation is based on the request.

11. A computing system comprising:
one or more processors;
one or more memory devices storing computer-executable instructions that, in response to execution by the one or more processors, cause the computing system to:
authenticate a user identifier via an authentication service;
based on authenticating the user identifier, cause output of a graphical user interface configured to elicit one or more responses associated with a user condition;
receive the one or more responses via the graphical user interface;
associate the one or more responses with the user identifier;
generate, based on the one or more responses, at least one of a visual representation or an aural representation indicative of the user condition; and
cause output of at least one of the visual representation or the aural representation.

12. The computing system of claim 11, wherein the user identifier is one or more of a username, a password, a generated data structure, or an access token.

13. The computing system of claim 11, wherein the user condition is one or more of a pre-operative condition, a post-operative condition, a mental health condition, a wellness screening, or a disease state.

14. The computing system of claim 11, wherein the graphical user interface is configured to elicit one or more responses associated with a user condition by presenting one or more queries associated with the user condition.

15. The computing system of claim 11, wherein associating the one or more responses with the user identifier comprises generating a data structure comprising a representation of each response of the one or more responses and a key value corresponding to the user identifier.

16. The computing system of claim 15, wherein the generating comprises determining, based on a quantity of the one or more responses and based on the representations of each response of the one or more responses, a graphical layout of the visual representation.

17. The computing system of claim 16, wherein the graphical layout comprises one or more of an object associated with each representation of each response of the one or more responses, a position of the object, time of the object, or a color of the object.

18. At least one computer-readable non-transitory medium having instructions encoded thereon that, in response to being executed, causes a computing system to:
authenticate a user identifier via an authentication service;
based on authenticating the user identifier, cause output of a graphical user interface configured to elicit one or more responses associated with a user condition;
receive the one or more responses via the graphical user interface;
associate the one or more responses with the user identifier;
generate, based on the one or more responses, at least one of a visual representation or an aural representation indicative of the user condition; and
cause output of at least one of the visual representation or the aural representation.

19. The at least one computer-readable non-transitory medium of claim 18, wherein the user condition is one or more of a pre-operative condition, a post-operative condition, a mental health condition, a wellness screening, or a disease state.

20. The at least one computer-readable non-transitory medium of claim 18, wherein the graphical user interface is configured to elicit one or more responses associated with a user condition by presenting one or more queries associated with the user condition.
